(19) United States
(12) Patent Application Publication
(10) Pub. No.: US 2006/0002970 A1
(43) Pub. Date: Jan. 5, 2006

(54) Method for coating a suture

(76) Inventors: Per Vilhelm Aspenberg, Linkoping (SE); Pentti Olavi Tengvall, Linkoping (SE); Bjorn Pasternak, Linkoping (SE)

Correspondence Address: FASTH LAW OFFICES (ROLF FASTH), 26 PINECREST PLAZA, SUITE 2, SOUTHERN PINES, NC, 28387-4301, US

(21) Appl. No.: 11/138,719

(22) Filed: May 26, 2005

Related U.S. Application Data
(63) Continuation-in-part of application No. 10/534,666, filed on May 11, 2005.

(51) Int. Cl. A61L 33/00; A61F 2/00

(52) U.S. Cl. 424/423; 427/2.24

(57) ABSTRACT

The method of the present invention is for coating a suture thread. The suture thread is coated with a protein film, such as fibrinogen. An active enzyme inhibitor, such as a matrix metallo proteinase inhibitor, is immobilized to or associated with the film.

### PRIOR APPLICATION

This is a continuation-in-part patent application of U.S. patent application Ser. No. 10/534,666, filed 11 May 2005 that claims priority from PCT/SE2004/001082 filed 1 Jul. 2004.

### TECHNICAL FIELD

The present invention relates to the coating of soft biomaterials, such as suture threads, intended for use in humans to inhibit tissue breakdown around the biomaterials. More particularly, the suture threads may be coated with a matrix metallo-proteinase inhibitor (MMP-inhibitor).

### BACKGROUND OF THE INVENTION

Collagen is the fundamental functional molecule in tissues like tendon and ligaments, and is largely responsible for the mechanical integrity of most tissues in the body, ranging from intestines to tendons. When a ligament or tendon has ruptured, or when other organs are operated on, the repair is often done with sutures. The suture threads have a grip in the collagenous substance of the tissue. The cells in the vicinity of the suture become activated by the trauma, either by the injury or by the suture itself. This activation leads to break-down of the tissue and is later followed by replacement with new collagen. It is well known that, for example, the repair of a ruptured tendon or an opened intestine with a suture will have a decreasing mechanical strength during the first days or weeks. Even though the suture fixation appears strong at the time of the operation, there is a high risk that the suture thread will cut through the tendon material when this has become softened in the days following the operation. This softening of the tissue around the sutures is a real problem that has troubled medical practitioners for a long time.

Matrix Metallo-Proteinases (MMPs) are a group of zinc-dependent enzymes responsible for the breakdown of collagen and other matrix molecules. MMPs are crucial in the turn-over of tissue matrix. A dramatic increase in the production and activation of MMPs is caused by injury or surgery on collagenous tissues. This leads to break-down of tissue and reduced strength following the suture procedure. This over-production of MMPs leads to break-down of tissue and reduced strength of the substances disposed around the sutures.

The suture threads themselves may also cause increased production of MMP. There is evidence that cells produce large amounts of MMPs specifically around sutures inserted into tendons. Additionally, unloading the tendon, such as in a cast after injury or surgery, can lead to dramatic deterioration of its mechanical properties within a short period of time. Immobilization, in both tendons and ligaments, leads to an increase in local MMP production. Thus, the weakening of tendons after injury, suture and unloading should principally be the result of increased MMP activity. The clinical problems corresponding to these phenomena are repair-site elongation, implying poorer healing, and, at worst, re-rupture.

Rates of re-rupture are significant after suturing intestines (intestinal anastomoses). Studies show that MMP production (expression) is high, especially at the suture line. It has recently been demonstrated that suturing of the bowel (colon) in rats is followed by a fast decrease in suture strength by about 50%. Systemic treatment of the rat with a metallo-proteinase inhibitor totally abolished this decrease in strength.

MMP inhibitors may be used to inhibit the breaking down of tissue caused by the MMPs. Skin wound healing can be accelerated by using MMP inhibitors. Animal models show that MMP-inhibitors enhance mechanical properties of (non-sutured) healing skin wounds. In some surgical situations, such as hernial surgery, a mesh of suture-like material is implanted to augment soft tissues and serve as a scaffold for scar formation. It is conceivable that there is a problem with local MMP production and activation adjacent to this net in the same way as has been shown for ordinary sutures.

Not only MMPs are activated in these situations. Cyclooxygenases are also activated which are enzymes responsible for the production of prostaglandins which are important for the development of inflammation. Inhibition of cyclooxygenases is done with common anti-inflammatory drugs. Such inhibition with systemic treatment improves the later phases of tendon repair.

Systemic medication, such as by administering substance in tablet or liquid form, implies drug interference with organs additional to those intended. Side-effects from systemic use of MMP-inhibitors for cancer therapy have been reported as well as from the antibiotic use of tetracyclines.

There is a need to inhibit the gradual weakening or softening of the tissue surrounding sutures that occurs after surgery. There is also a need to prevent the side effects of systemic distribution of the relevant drug to inhibit tissue weakening around the sutures.

### SUMMARY OF THE INVENTION

The method and the coated suture of the present invention provide a solution to the above-outlined problems. The implementation of the present invention diminishes the decrease in tissue mechanical strength that is the result of injury, surgery and suturing. The method of the present invention is for coating pharmacological substances that inhibit tissue break-down onto a suture thread.

More particularly, the present invention provides means for inhibiting tissue weakening. At the same time, the adverse effects of systemic distribution of the relevant medical substances are avoided. One important feature of the present invention is that a local drug delivery is achieved through the attachment of the relevant medical drug to the device left in the body after a surgery or other intervention. Another important feature is to provide sustained medical treatment rather than merely a single dosage. Preferably, there is a gradual release of the locally delivered drug at the site of the surgery.

### DETAILED DESCRIPTION

The present invention includes the coating of suture threads, and other devices to be left in soft tissue, with enzyme inhibitors such as MMP-inhibitors or other substances that interfere with collagen and tendon tissue to contribute to the maintained integrity of collagen and tendon tissue. Preferably, the MMP inhibitors should be gradually released to reduce the effectiveness of MMP.

In general, MMP is over-produced in areas of injury or surgery so that the amount of tissue that is broken down exceeds the reproduction of tissue. By gradually releasing MMP inhibitors there is a better balance created in the area of injury between the breaking down of tissue by MMP and new reproduction of tissue. In this way, suture threads and other biomaterials may be more effectively used in areas of injury or surgery since the tissue surrounding the suture threads is not broken down as much or not at all. More particularly, the MMP inhibitor should be gradually released as long as there is an injury so as to inhibit the effectiveness of the overproduced MMP during this time. When the injury has healed there is no need to inhibit MMP and the natural balance between the breaking down of tissue and regeneration of tissue is reestablished.

Adherence of the drug substance, containing MMP-inhibitors, to the suture or other device is achieved by adsorption, covalent binding, electrostatic interactions or any other suitable mechanism. Preferably, the adherence is achieved through the formation of a fibrinogen, matrix or matrix formed by any other suitable protein, peptide, or substance, and the binding of MMP-inhibitor (or other substance) to the matrix to reduce the effect of MMP around the suture.

In this way, the MMP inhibitor coated suture has a matrix, for example, a fibrinogen matrix, attached to it in which the MMP-inhibitor is associated. The matrix may be composed of several layers of protein covalently bound to each other, and the bottom layer may be attached via covalent binding, van der Waals, hydrophobic, coulombic and/or other suitable interactions or bonding methods, to the suture material. Preferably, a first amount of the MMP inhibitor is covalently bound to the matrix. However, other ways of binding the inhibitor may be used such as electro-statical binding or hydrophobic attachment of the inhibitor. A combination of covalent and electrostatic loading may be preferable to obtain a gradual release of the MMP inhibitor. Preferably, a second amount of the MMP inhibitor is adhered to the matrix for easy release. The gradual release of the MMP inhibitor from the coated suture thread decreases the local MMP activity and thus preserves the mechanical strength of the collagenous material surrounding the suture thread and decreases the risk of failure of the suture. A COX-inhibitor could serve the same purpose, but acting in an earlier step, i.e. reducing the induction of MMP-producing activity of inflammatory and tissue cells.

The MMP inhibitor can be adhered directly onto the suture. If incorporated in a matrix adhered to the suture, the release velocity and thus pharmacological dose can be controlled. Additionally, more substance per area suture can be delivered by using the matrix. This should be done in a way that permits a controlled release of the MMP inhibitor that last several days or even a few weeks. The incorporation of the drug within the substance of a resorbable thread, such as a PLGA suture, releases most of the drug too late, when the suture is losing strength in itself and the surrounding tissue start to soften before it is exposed to the MMP inhibitor.

Preferably, the method of the present invention should be applied primarily on tendon and during intestine surgery. Other applications are also possible. For example, the method of the present invention may be used for simple skin suturing. Additionally, the suture threads coated may be of a resorbable material.

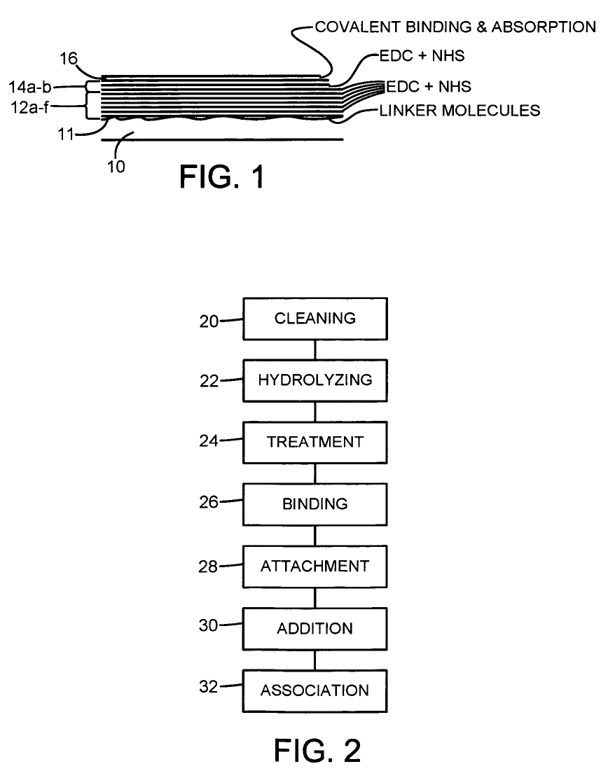

FIG. 1 is a schematic cross-sectional view of the coated suture. Preferably, the suture thread 10 has a linker molecule layer 11 bound to the surface of the suture. A plurality of protein layers 12 such as fibrinogen layers are then applied on top thereof. A free carboxyl terminal of the first protein layer 12a may be activated by for example a carbodiimide, such as ethyl-dimethyl-aminopropylcarbodiimide (EDC), and hydroxy-succinimide (NHS) to attract and by peptide bond formation capture more protein so as to form a second protein layer 12b. The EDC activates the carboxyl groups, of the first protein layer, so that amino groups of the protein in solution may be chemically bound thereto. By repetition of the EDC/NHS activation procedure, a plurality of protein layers may be immobilized and cross-linked, so forming a matrix structure. The total thickness of the protein layers 12 may be increased by increasing the number of layers. The top layer 16 preferably include the enzyme inhibitor, such as an MMP inhibitor, so that a first amount of the inhibitor is covalently, or by other mechanisms firmly, bound to the protein and a second amount of the inhibitor is adhered to the protein by for example, absorption or any other mechanism which makes the release relatively easy.

FIG. 2 illustrates some of the steps used to coat the suture. In a first cleaning step 20, the suture is cleaned in for example a suitable alcohol. The suture surface is then hydrolyzed in a hydrolyze step 22. Charged and chemically reactive groups are created on the suture surface in a treatment step 24. In a binding step 26, a suitable linker molecule is then chemically bound to the suture surface. In an attachment step 28, the first protein layer is chemically attached to the linker molecule. In an adding step 30, a plurality of protein layers are added according to the principles described above. In an association step 32, a first amount of a suitable enzyme inhibitor is chemically bound to the protein layers by the EDC/NHS coupling chemistry described, or any other suitable method. A second amount of the enzyme inhibitor is absorbed in the matrix structure formed by the protein layers. An example of the details of each step is illustrated in the example below.

### EXAMPLE

Suture materials made of e.g. polyamides such as nylon-6,6 and nylon-6, or poly(p-dioxanone) or polylactide/-glycolide, are cleaned according to standard laboratory practice for 10 minutes by incubation in 70% ethanol followed by copious rinsing in distilled water and dried in nitrogen gas followed by 30 seconds exposure to UV. The structure surfaces become hydrolyzed during typically 3 hours in distilled water and treated one minute in a Radio Frequency Plasma chamber. Radio frequency plasma treatment generates charged and chemically reactive surface groups onto which for example spacers or proteins can be covalently attached. For example, surface carboxyl or amine groups may be formed on the suture via the surface activation procedures.

Thereafter, a linker molecule such as glutaraldehyde or ethyl-dimethyl-aminopropylcarbodiimide (EDC) is bound to the surface. One layer of fibrinogen from 1 mg/ml solution, or another macromolecule, becomes covalently attached by the assistance of the linker molecule. More fibrinogen or another macromolecule may subsequently be bound to this first layer in order to create a controllable but thin (thickness less than one micrometer) matrix into which the drug can be attached and/or associated.

Sutures with ten layers of fibrinogen may be prepared in the following way. Sutures prepared as above are then incubated for thirty minutes in 1 mg/ml protein dissolved in phosphate buffered saline (PBS) at pH 7.4. The specimen surfaces are thereafter extensively rinsed in PBS and incubated for thirty minutes in PBS at pH 5.5, containing 0.2M ethyl-dimethyl-aminopropylcarbodiimide (EDC). The specimen surfaces are again incubated for thirty minutes in a newly made 1-mg/ml protein solution in PBS, pH 5.5, thereafter rinsed in the PBS buffer and again incubated in the EDC/NHS solution. This procedure is repeated ten times to produce the ten-layer fibrinogen coating but is not limited to this number of protein incubations. Since the EDC/NHS solution is unstable at room conditions, new solutions are prepared every second hour.

The MMP-inhibitor, e.g. a tetracycline, is immobilized to the fibrinogen multiplayer using the above-described EDC/NHS coupling technique. The suture specimens are stored in a solution of the same or a different MMP-inhibitor for up to 24 hours to allow additional loading of the matrix with loosely bound substance. The specimens are removed from the solution, blown dry in nitrogen, and kept sealed at ambient until used.

The thickness of the cross-linked fibrinogen layer is approximately 280 Angstroms and the MMP-inhibitor layers between 5 and 100 Angstroms. The MMP-inhibitor coated suture interferes with MMP at the surgical site, lowering the activity of the latter. The gradual release of the MMP-inhibitor provides a sustained effect resulting in maintained integrity of the otherwise degenerated tissue, for example collagen and tendon that surrounds the suture threads

While the present invention has been described in accordance with preferred compositions and embodiments, it is to be understood that certain substitutions and alterations may be made thereto without departing from the spirit and scope of the following claims.

1. A method for coating surgical sutures, comprising:
providing a suture for repairing areas of damaged tissue;
coating the suture with an enzyme inhibitor;
the suture releasing the enzyme inhibitor; and
the enzyme inhibitor inhibiting an enzyme from breaking down tissue surrounding the suture.

2. The method according to claim 1 wherein the method further comprises coating with MMP inhibitor to inhibit MMP from breaking down tissue.

3. The method according to claim 1 wherein the method further comprises coating the suture with a COX inhibitor to inhibit production of MMP.

4. The method according to claim 2 wherein the method further comprises chemically binding the MMP inhibitor to the suture.

5. The method according to claim 1 wherein the method further comprises forming fibrinogen layers on the suture.

6. The method according to claim 1 wherein the method further comprises using a tetracycline substance as the enzyme inhibitor.

7. The method according to claim 1 wherein the method further comprises exposing the suture to radio frequency plasma treatment to generate a chemically reactive surface group on a surface of the suture.

8. The method according to claim 1 wherein the method further comprises chemically binding a linker molecule to the suture.

9. The method according to claim 8 wherein the method further comprises covalently binding a layer to the linker molecule.

10. The method according to claim 1 wherein the method further comprises forming a matrix coating of fibrinogen and an MMP inhibitor.

11. The method according to claim 2 wherein the method further comprises covalently binding a first amount of MMP inhibitor to a fibrinogen layer

12. The method according to claim 11 wherein the method further comprises the fibrinogen layer absorbing a second amount of MMP inhibitor without a covalent binding.
